(19) United States
(12) Patent Application Publication
(10) Pub. No.: US 2017/0143600 A1
(43) Pub. Date: May 25, 2017

(54) AGENTS AND METHOD FOR THE TEMPORARY SHAPING OF KERATIN-CONTAINING FIBERS

(71) Applicant: Henkel AG & Co. KGaA, Duesseldorf (DE)

(72) Inventors: Anna Puls, Winsen (DE); Sandra Fuchs, Pinneberg (DE); Nora Koopmann, Hamburg (DE)

(73) Assignee: Henkel AG & Co. KGaA, Duesseldorf (DE)

(21) Appl. No.: 15/353,975

(22) Filed: Nov. 17, 2016

(30) Foreign Application Priority Data
Nov. 25, 2015 (DE) 10 2015 223 348.0

(51) Int. Cl. A61K 8/25; A61Q 5/06; A61K 8/34; A61K 8/06; A61K 8/29; A61K 8/92

(52) U.S. Cl. CPC A61K 8/25; A61K 8/29; A61K 8/92; A61K 8/925; A61K 8/345; A61K 8/06; A61Q 5/06; A61K 2800/43

(57) ABSTRACT

Cosmetic compositions including
a) 0.05 to 10% by weight of a hydrophobized metal oxide powder,
b) 0.01 to 2.0% by weight of at least one white pigment,
c) 1.0 to 50% by weight of wax,
d) 1.0 to 30% by weight of an emulsifier,
e) 0.1 to 25% by weight of an organic polyol, and
f) 5.0 to 90% by weight of water,
   the use thereof for temporary hair shaping, and a method using these compositions.

### FIELD OF THE INVENTION

The present invention generally relates to the technical field of the temporary shaping of keratin-containing fibers, in particular human hair.

### BACKGROUND OF THE INVENTION

Styling agents for shaping keratin-containing fibers have been known for some time and are used in various forms for volumizing, refreshing, and retaining hairstyles, which for many hair types can only be achieved using setting active substances. Both hair treatment agents used to shape hair permanently and those that shape it temporarily play an important role in this respect.

Sprayable products as well as product forms that are worked into the hairstyle with the aid of a comb or with the fingers are suitable for the temporary shaping of keratin-containing fibers. The last-mentioned product group comprises oils as well as gels, creams, and powders. Powdered styling agents in particular are enjoying increasing popularity because of their easy dispensability and their cosmetic properties such as a high hairstyle hold, increase in volume, remodelability, and their matting effect.

German patent application DE 102008057261 A1 therefore describes powdered compositions which are used for the temporary reshaping of hair for a very strong hold of the set hairstyle.

The subject of international application WO 2007/051511 A1 is the use of a powdered composition, including 50 to 95% by weight of an aqueous solvent, hydrophobized silicon dioxide powder, and a film-forming and/or setting polymer, present at least in the aqueous solvent, for the temporary shaping of keratinic fibers.

The powdered hair cosmetics of the state of the art meanwhile do in fact provide a good hold and high volume, and are notable for good dispensability. The matting effect associated with the use of these agents, however, does not satisfy the wish of all consumers and is replaced, at least partially, by the wish for hair care and hair shine.

The object of the present invention, therefore, was to provide hair treatment agents for the temporary shaping, which are notable for an increased hair shine, apart from a good hairstyle hold, high hair volume, and remodelability. The agents should be usable, moreover, on wet and on dry hair.

It was established that these objects can be achieved with the aid of a complex active substance mixture, to which, apart from other components, inter alia, a hydrophobized metal oxide powder, usually used in powdered styling agents, is added.

Furthermore, other desirable features and characteristics of the present invention will become apparent from the subsequent detailed description of the invention and the appended claims, taken in conjunction with this background of the invention.

### BRIEF SUMMARY OF THE INVENTION

Cosmetic compositions including

a) 0.05 to 10% by weight of a hydrophobized metal oxide powder,

b) 0.01 to 2.0% by weight of at least one white pigment,

c) 1.0 to 50% by weight of wax,

d) 1.0 to 30% by weight of an emulsifier,

e) 0.1 to 25% by weight of an organic polyol, and

f) 5.0 to 90% by weight of water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention constitutes cosmetic compositions, including

a) 0.05 to 10% by weight of a hydrophobized metal oxide powder,

b) 0.01 to 2.0% by weight of at least one white pigment,

c) 1.0 to 50% by weight of wax,

d) 1.0 to 30% by weight of an emulsifier,

e) 0.1 to 25% by weight of an organic polyol, and

f) 5.0 to 90% by weight of water.

The agents of the invention are preferably present in the form of creams or pastes. The viscosity of agents of the invention is preferably between 70,000 and 600,000 mPas, preferably between 100,000 and 400,000 mPas (20° C., rotational viscometer DV 2T “Brookfield,” spindle TE, 5 rotations per minute, and Helipath).

A first essential component of the compositions of the invention is the hydrophobized metal oxide powder. Preferred compositions are characterized in that they include the hydrophobized metal oxide powder, based on their total weight, in amounts of 0.1 to 10% by weight, preferably of 0.2 to 8.0% by weight, and in particular of 0.5 to 4.0% by weight. The optimal amount in this case depends, in addition to the weight proportions of the other components, primarily on the hydrophobicity of the employed silicon dioxide powder.

Metal oxides that have been modified at least on the surface of the particles such that the modified particle is wetted less by water than the unmodified particle are to be understood as hydrophobized in the context of the invention.

The particle diameter of the primary particles of preferred hydrophobized metal oxides is preferably less than 5 μm, particularly preferably less than 1 μm, and in particular between 1 and 50 nm.

At least one representative of the group formed by silanes, halo silanes, alkoxysilanes, and silazanes is preferably suitable according to the invention as a reagent for silanizing the metal oxide. Preferably suitable hydrophobized metal oxides of the hydrophobized metal oxide powder are selected according to the invention from at least one representative of the group formed by hydrophobized silicates, hydrophobized aluminosilicates, hydrophobized titanium dioxide, and hydrophobized silicon dioxide. Hydrophobized silicates have proven to be particularly suitable for preparing the cosmetic compositions of the invention, pyrogenic silicic acid aftertreated by silanization or by reaction with polydimethylsiloxane having particular advantages.

Particularly preferably, the powdered composition of the invention includes as the hydrophobized metal oxide powder at least hydrophobized silicon dioxide. Preferred furthermore are hydrophobized silicon dioxides that have a specific BET surface between 10 and 400 m²/g, preferably between 40 to 300 m²/g, and in particular 80 to 150 m²/g.

Particularly preferably, the powdered composition of the invention includes as the hydrophobized metal oxide powder at least silanized, hydrophobized silicon dioxide. Preferably, at least one representative of the group formed by silanes, halosilanes, alkoxysilanes, and silazanes is suitable according to the invention as a reagent for silanizing the silicon dioxide.

Preferred representatives of the group of silanes are hexa(C₁-C₂₀) alkyl disilanes, particularly hexamethyldisilane.

If a halosilane is used as the silylating agent, selected as the preferred halosilane is at least one compound from the group formed by the compounds

[(C₁-C₂₀)alkyl]_{z′}SiX_{(4-z′)}

X₃Si[(CH₂)ₙ—R]

X₂[(C₁-C₂₀)alkyl]Si(CH₂)ₙ—R

[(C₁-C₂₀)alkyl]_{(y′+1)}[R—(CH₂)ₙ]_{(2-y′)}SiX,

where
X denotes a chlorine, bromine, or iodine atom,
z′ is a number 1, 2, or 3,
y′ is a number 0, 1, or 2,
n is an integer from 1 to 20, and
R stands for a group from
C₁-C₁₀)alkyl-, aryl-, (C₁-C₆)perfluoroalkyl-, —NH₂, —N₃, —SCN, —CH═CH₂, —O(O)C—C(CH₃)═CH₂, —OCH₂—CH═CH₂,

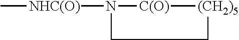

—NH—C(O)O-Me, —NH—C(O)O-Et, —NH—(CH₂)₃—Si(O(C₁-C₆)alkyl)₃.

If an alkoxysilane is used as the silylating agent, selected as the preferred alkoxysilane is at least one compound from the group formed by the compounds

[(C₁-C₂₀)alkylO]_{z}Si(C₁-C₂₀)alkyl_{(4-z)}

[(C₁-C₂₀)alkylO]_{z}Si[(CH₂)ₙ—R]_{(4-z)}

[(C₁-C₂₀)alkylO]₂[(C₁-C₂₀)alkyl]Si(CH₂)ₙ—R

[(C₁-C₂₀)alkylO][(C₁-C₂₀)alkyl]₂Si(CH₂)ₙ—R

[(C₁-C₂₀)alkylO][(C₁-C₂₀)alkyl]Si[(CH₂)ₙ—R]₂

(C₁-C₂₀alkyl)₃SiO—C(CH₃)═N—Si(C₁-C₂₀)alkyl₃,

where
n is an integer from 1 to 20, and
z denotes a number 1, 2, or 3,
R stands for a group from
(C₁-C₂₀)alkyl-, aryl-, (C₁-C₆)perfluoroalkyl-, —NH₂, —N₃, —SCN, —CH═CH₂, —O(O)C—C(CH₃)═CH₂, —OCH₂—CH═CH₂,

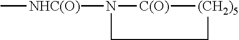

—NH—C(O)O-Me, —NH—C(O)O-Et, —NH—(CH₂)₃—Si(O(C₁-C₆)alkyl)₃.

Selected as the preferred silazane is at least one compound from the class of disilazanes, in particular at least one compound from disilazanes of the formula

R′₂R″Si—NH—SiR′₂R″

where
R′ denotes a (C₁-C₂₀) alkyl group and
R″ denotes a (C₁-C₂₀) alkyl group or a vinyl group. A particularly preferred silazane is hexamethyldisilazane.

All of the aforesaid alkyl groups, whether (C₁-C₆) alkyl, (C₁-C₁₀) alkyl, or (C₁-C₂₀) alkyl, can be both cyclic and linear or branched. Examples of alkyl groups usable according to the invention are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group of the invention is the phenyl group.

Examples of a (C₁-C₆) perfluoroalkyl group of the invention are trifluoromethyl, perfluoroethyl, perfluoropropyl, and perfluorohexyl.

Hydrophobized silicon dioxides obtained by silanization of pyrogenic silicon dioxide are preferably used.

Silanized, hydrophobized silicon dioxides are selected particularly preferably from at least one compound of the group formed by trimethyl silylate-coated silicon dioxide, dimethyl silylate-coated silicon dioxide, and octyl silylate-coated silicon dioxide.

Hydrophobized silicon dioxides obtained by silanization of pyrogenic silicon dioxide are preferably used. Compositions that include as the hydrophobized metal oxide powder a hydrophobized silicate obtained by silanization of pyrogenic silicon dioxide are preferred because of their product properties, in particular their storage stability and their cosmetic action.

A variety of suitable hydrophobized silicon dioxides are commercially available. Recited as examples are Aerosil® R104 V, Aerosil® R106, Aerosil® R202, Aerosil® R805, Aerosil® R812, Aerosil® R812S, Aerosil® R972, and Aerosil® R8200, all from Degussa, and HDK® H2000, HDK® H2050, and HDK® H3004, all from Wacker.

It is particularly preferred to use hydrophobized silicon dioxides that are obtainable under the names Aerosil® R202, Aerosil® R812S, or Aerosil® R972. It is very particularly preferred to use the silicon dioxide with the INCI name Silica Silylate, which is marketed by the company Degussa under the name Aerosil® R812S.

Aftertreatment with polydimethylsiloxane offers an alternative to the hydrophobizing by silanization. Suitable compositions of the invention are those that include pyrogenic silicic acid aftertreated with polydimethylsiloxane. Suitable metal oxides with the INCI name “Silica Dimethicone Silylate” are marketed, for example, by the company Evonik under the trade name Aerosil® R202.

A second essential component of the cosmetic compositions of the invention is white pigment b). White pigments are achromatic inorganic pigments with a high refractive index (preferably greater than 1.8), which are usually produced synthetically and are used primarily for creating optical whiteness in coating compositions or as fillers in, e.g., plastics. Titanium dioxide primarily can be used particularly preferably according to the invention; in addition, zinc oxide, zinc sulfide, lithopone, and aluminum starch octenylsuccinate, rather uncommon for the technical purposes, have proven suitable according to the invention.

The weight proportion of the white pigment in the total weight of the cosmetic compositions is preferably 0.02 to 1.5% by weight, preferably of 0.04 to 1.2% by weight, and in particular of 0.05 to 1.0% by weight.

The degree of hold of the temporarily shaped keratinic fibers is increased by the addition of the white pigment. The shine of the hair is reduced. Moreover, the texture and feel of the hair are improved.

The compositions of the invention include a wax c) as the third essential component. The amount of the wax employed, based on the total weight of the compositions, is preferably 2.0 to 40% by weight, preferably 4.0 to 30% by weight, and in particular 5.0 to 25% by weight.

Preferred waxes have a melting point in the range of 40° C. to 90° C., particularly preferably in the range of 50° C. to 85° C., and in particular in the range of 50° C. to 75° C.

All waxes can be used in principle that melt in the mentioned temperature range and are physiologically compatible. Waxes particularly preferred according to the invention are beeswax, carnauba wax, candelilla wax, montan wax, and microcrystalline waxes, but preferably beeswax.

The teaching of the invention also encompasses the combined use of multiple waxes. Thus, the addition of small amounts of carnauba wax can be used to increase the melting and drop point of another wax and to reduce its tackiness. Furthermore, a number of wax mixtures, optionally mixed with further additives, are also commercially available. Those having the names “Spezialwachs 7686 OE” (a mixture of cetyl palmitate, beeswax, microcrystalline wax, and polyethylene with a melting range of 73-75° C.; manufacturer: Kahl & Co), Polywax® GP 200 (a mixture of stearyl alcohol and polyethylene glycol stearate with a melting point of 47-51° C.; manufacturer: Croda), and “Weichceresin® FL 400” (a vaseline/vaseline oil/wax mixture with a melting point of 50-54° C.; manufacturer: Parafluid Mineralölgesellschaft) are examples of mixtures employed preferably according to the invention.

It was possible to achieve an unexpected increase in the volume of the temporarily shaped keratinic fibers by the addition of wax to the cosmetic formulations used according to the invention.

The fourth essential component of the cosmetic compositions of the invention is emulsifier d). The weight proportion of emulsifier d) in terms of the total weight of the compositions is preferably 1.2 to 25% by weight, preferably 1.5 to 20% by weight, and in particular 2.0 to 15% by weight.

The cosmetic agents preferably include furthermore at least one representative from the group of anionic, amphoteric, zwitterionic, nonionic, and cationic emulsifiers or mixtures thereof. Used with preference are emulsifiers that are solid at room temperature (20° C.), in particular nonionic emulsifiers that are solid at room temperature (20° C.).

Particularly suitable as nonionic emulsifiers are fatty acid partial glycerides, which are to be understood to be monoglycerides, diglycerides, and technical mixtures thereof. When technical products are used, small amounts of triglycerides can be present as a result of the manufacturing. The partial glycerides preferably follow the formula:

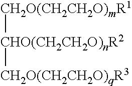

where R¹, R², and R³ independently of one another stand for hydrogen or for a linear or branched, saturated and/or unsaturated acyl group having 6 to 22, preferably 12 to 18, carbon atoms with the proviso that at least one of these groups stands for an acyl group, and at least one of these groups for hydrogen. The sum (m+n+q) stands for 0 or for numbers from 1 to 100, preferably for 0 or 5 to 25. R¹ preferably stands for an acyl group, and R² and R³ for hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, and technical mixtures thereof. Oleic acid monoglycerides are used with preference.

The surface-active, medium-chain fatty alcohols, in particular cetyl alcohol and stearyl alcohol, form a further group of preferred emulsifiers d).

Particularly preferred emulsifiers d) are selected
i) from the group of fatty acid partial glycerides, preferably from the group of glycerol monostearate and glycerol distearate,
ii) from the group of surface-active, medium-chain fatty alcohols, preferably from the group of cetyl alcohol and stearyl alcohol

Very particularly preferred is the use of an emulsifier combination of fatty acid partial glycerides and surface-active, medium-chain fatty alcohols, preferably a combination of glycerol monostearate and glycerol distearate with cetyl alcohol and stearyl alcohol, in particular a combination of glycerol monostearate and glycerol distearate with cetyl alcohol and stearyl alcohol.

The compositions of the invention include an organic polyol as the fifth essential component. Preferred compositions include organic polyol e), based on their total weight, in amounts of 0.5 to 20, preferably 1.0 to 15% by weight, and in particular 2.0 to 10% by weight.

The polyol can be used as an individual substance or in the form of polyol mixtures. Preferred agents of the invention are notable in that they include less than four, preferably one to three, but in particular only one polyol. Suitable in this case for preparing cosmetic compositions are in particular polyols from the group comprising glycerol, 1,2-ethanediol, polyethylene glycols with MW>400, propanediol, butanediol, particularly 1,3-butanediol, hexanediol, particularly 1,6-hexanediol, sorbitol, threitol, erythritol, arabitol, altritol, ribitol, xylitol, galactitol, mannitol, iditol, and panthenol. Particularly suitable organic polyols are glycerol, sorbitol, and panthenol. Preferred compositions of the invention are characterized in that they include as organic polyol e) at least one polyol from the group comprising glycerol, polyethylene glycol, propylene glycol, butylene glycol, and hexanediol, preferably glycerol.

The addition of polyol e) improves the properties of the compositions of the invention during storage and application and has advantageous cosmetic effects.

The weight proportion of polyol e) in terms of the total weight of compositions of the invention can vary over broad ranges. In the compositions of the invention with a low polyol content, a greater amount of water is added preferably to the compositions by way of compensation. The water content of compositions of the invention in this case is preferably 10 to 85% by weight, preferably 40 to 85% by weight, and in particular 65 to 82% by weight.

In summary, preferred compositions of the invention include

a) 0.5 to 4.0% by weight of a hydrophobized metal oxide powder,

b) 0.05 to 1.0% by weight of at least one white pigment,

c) 5.0 to 25% by weight of wax,

d) 2.0 to 15% by weight of an emulsifier,

e) 2.0 to 10% by weight of an organic polyol, and

f) 65 to 82% by weight of water.

A specific embodiment of a preferred composition of this kind of the invention includes
a) 0.5 to 4.0% by weight of a hydrophobized silicate obtained by silanization of pyrogenic silicon dioxide,
b) 0.05 to 1.0% by weight of titanium dioxide,
c) 5.0 to 25% by weight of beeswax,
d) 2.0 to 15% by weight of an emulsifier,
e) 2.0 to 10% by weight of glycerol, and
f) 65 to 82% by weight of water.

The cosmetic compositions based on their total weight include 0.1 to 10% by weight of homo- and/or copolymer g) as a particularly preferred optional component. Preferred compositions are characterized in that they include the homo- and/or copolymer b), based on their total weight, in amounts of 0.2 to 10% by weight, preferably of 0.4 to 6.0% by weight, and in particular of 0.8 to 4.0% by weight.

Nonionic polymers are used with particular preference as homo- and/or copolymers b). Suitable nonionic homo- and/or copolymers b) are, for example:
Polyvinylpyrrolidones, as are marketed, for example, under the name Luviskol® (BASF), are preferred homopolymers b).
Vinylpyrrolidone/vinyl ester copolymers, as they are marketed, for example, under the trademark Luviskol® (BASF). Luviskol® VA 64 and Luviskol® VA 73, each being vinylpyrrolidone/vinyl acetate copolymers, are preferred nonionic copolymers b).
Cellulose ethers, such as hydroxypropyl cellulose, hydroxyethyl cellulose, and methyl hydroxypropyl cellulose.

Preferred compositions include polyvinylpyrrolidone and/or vinylpyrrolidone/vinyl acetate copolymer, preferably polyvinylpyrrolidone, as homo- and/or copolymer b).

The composition of some preferred cosmetic agents can be obtained from the following tables (data are given in percentages by weight, based on the total weight of the cosmetic agent, unless otherwise stated).

| | Formula | Formula | Formula | Formula | Formula |
|---|---|---|---|---|---|
| | 1 | 2 | 3 | 4 | 5 |
| Hydrophobized | 0.05 to 10 | 0.1 to 10 | 0.2 to 8.0 | 0.2 to 6.0 | 0.5 to 4.0 |
| silicon dioxide | | | | | |
| White pigment | 0.01 to 2.0 | 0.01 to 2.0 | 0.01 to 1.5 | 0.04 to 1.2 | 0.05 to |
| | | | | | 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 6 | 7 | 8 | 9 | 10 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 11 | 12 | 13 | 14 | 15 |
| Hydrophobized | 0.05 to 10 | 0.1 to 10 | 0.2 to 8.0 | 0.2 to 6.0 | 0.5 to 4.0 |
| silicon dioxide | | | | | |
| White pigment | 0.01 to 2.0 | 0.01 to 2.0 | 0.01 to 1.5 | 0.04 to 1.2 | 0.05 to |
| | | | | | 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 16 | 17 | 18 | 19 | 20 |
| Hydrophobized | 0.05 to 10 | 0.1 to 10 | 0.2 to 8.0 | 0.2 to 6.0 | 0.5 to 4.0 |
| silicon dioxide | | | | | |
| White pigment | 0.01 to 2.0 | 0.01 to 2.0 | 0.01 to 1.5 | 0.04 to 1.2 | 0.05 to |
| | | | | | 1.0 |
| Wax | 1.0 to 50 | 2.0 to 40 | 2.0 to 40 | 4.0 to 30 | 5.0 to 25 |
| Fatty acid | 1.0 to 30 | 1.2 to 25 | 1.5 to 20 | 2.0 to 15 | 2.0 to 15 |
| partial | | | | | |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 21 | 22 | 23 | 24 | 25 |
| Hydrophobized | 0.05 to 10 | 0.1 to 10 | 0.2 to 8.0 | 0.2 to 6.0 | 0.5 to 4.0 |
| silicon dioxide | | | | | |
| White pigment | 0.01 to 2.0 | 0.01 to 2.0 | 0.01 to 1.5 | 0.04 to 1.2 | 0.05 to |
| | | | | | 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 26 | 27 | 28 | 29 | 30 |
| Hydrophobized | 0.05 to 10 | 0.1 to 10 | 0.2 to 8.0 | 0.2 to 6.0 | 0.5 to 4.0 |
| silicon dioxide | | | | | |
| White pigment | 0.01 to 2.0 | 0.01 to 2.0 | 0.01 to 1.5 | 0.04 to 1.2 | 0.05 to |
| | | | | | 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 31 | 32 | 33 | 34 | 15 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 36 | 37 | 38 | 39 | 40 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| Fatty acid | 1.0 to 30 | 1.2 to 25 | 1.5 to 20 | 2.0 to 15 | 2.0 to 15 |
| partial | | | | | |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 41 | 42 | 43 | 44 | 45 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 46 | 47 | 48 | 49 | 40 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 41 | 42 | 43 | 44 | 45 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Beeswax | 1.0 to 50 | 2.0 to 40 | 2.0 to 40 | 4.0 to 30 | 5.0 to 25 |
| Fatty acid | 1.0 to 30 | 1.2 to 25 | 1.5 to 20 | 2.0 to 15 | 2.0 to 15 |
| partial | | | | | |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 46 | 47 | 48 | 49 | 50 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Beeswax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 51 | 52 | 53 | 54 | 55 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 56 | 57 | 58 | 59 | 60 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| Fatty acid | 1.0 to 30 | 1.2 to 25 | 1.5 to 20 | 2.0 to 15 | 2.0 to 15 |
| partial | | | | | |
| glyceride | | | | | |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula | Formula | Formula | Formula | Formula |
|---|---|---|---|---|---|
| | 61 | 62 | 63 | 64 | 65 |
| Hydrophobized | 0.05 to 10 | 0.1 to 10 | 0.2 to 8.0 | 0.2 to 6.0 | 0.5 to 4.0 |
| silicon dioxide | | | | | |
| Titanium | 0.01 to 2.0 | 0.01 to 2.0 | 0.01 to 1.5 | 0.04 to 1.2 | 0.05 to |
| dioxide | | | | | 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 66 | Formula 67 | Formula 68 | Formula 69 | Formula 70 |
|---|---|---|---|---|---|
| Silica silylate | 0.05 to 10 | 0.1 to 10 | 0.2 to 8.0 | 0.2 to 6.0 | 0.5 to 4.0 |
| White pigment | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 71 | Formula 72 | Formula 73 | Formula 74 | Formula 75 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 76 | Formula 77 | Formula 78 | Formula 79 | Formula 80 |
|---|---|---|---|---|---|
| Silica silylate | 0.05 to 10 | 0.1 to 10 | 0.2 to 8.0 | 0.2 to 6.0 | 0.5 to 4.0 |
| White pigment | 0.01 to 2.0 | 0.01 to 2.0 | 0.01 to 1.5 | 0.04 to 1.2 | 0.05 to 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 81 | Formula 82 | Formula 83 | Formula 84 | Formula 85 |
|---|---|---|---|---|---|
| Silica silylate | 0.05 to 10 | 0.1 to 10 | 0.2 to 8.0 | 0.2 to 6.0 | 0.5 to 4.0 |
| White pigment | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Fatty acid partial | 1.0 to 30 | 1.2 to 25 | 1.5 to 20 | 2.0 to 15 | 2.0 to 15 |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 86 | Formula 87 | Formula 88 | Formula 89 | Formula 90 |
|---|---|---|---|---|---|
| Silica silylate | 0.05 to 10 | 0.1 to 10 | 0.2 to 8.0 | 0.2 to 6.0 | 0.5 to 4.0 |
| White pigment | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 91 | Formula 92 | Formula 93 | Formula 94 | Formula 95 |
|---|---|---|---|---|---|
| Silica silylate | 0.05 to 10 | 0.1 to 10 | 0.2 to 8.0 | 0.2 to 6.0 | 0.5 to 4.0 |
| White pigment | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 96 | Formula 97 | Formula 98 | Formula 99 | Formula 100 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 101 | Formula 102 | Formula 103 | Formula 104 | Formula 105 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Fatty acid partial | 1.0 to 30 | 1.2 to 25 | 1.5 to 20 | 2.0 to 15 | 2.0 to 15 |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 106 | Formula 107 | Formula 108 | Formula 109 | Formula 110 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 111 | Formula 112 | Formula 113 | Formula 114 | Formula 115 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 116 | Formula 117 | Formula 118 | Formula 119 | Formula 120 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Beeswax | 1.0 to 50 | 2.0 to 40 | 2.0 to 40 | 4.0 to 30 | 5.0 to 25 |
| Fatty acid partial | 1.0 to 30 | 1.2 to 25 | 1.5 to 20 | 2.0 to 15 | 2.0 to 15 |
| glyceride | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 121 | Formula 122 | Formula 123 | Formula 124 | Formula 125 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Beeswax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Organic polyol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 126 | Formula 127 | Formula 128 | Formula 129 | Formula 130 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Beeswax | 1.0 to 50 | 2.0 to 40 | 2.0 to 40 | 4.0 to 30 | 5.0 to 25 |
| Emulsifier | 1.0 to 30 | 1.2 to 25 | 1.5 to 20 | 2.0 to 15 | 2.0 to 15 |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 131 | Formula 132 | Formula 133 | Formula 134 | Formula 135 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| Fatty acid partial | 1.0 to 30 | 1.2 to 25 | 1.5 to 20 | 2.0 to 15 | 2.0 to 15 |
| glyceride | | | | | |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

| | Formula 136 | Formula 137 | Formula 138 | Formula 139 | Formula 140 |
|---|---|---|---|---|---|
| Silica silylate | 0.05 to 10 | 0.1 to 10 | 0.2 to 8.0 | 0.2 to 6.0 | 0.5 to 4.0 |
| Titanium dioxide | 0.01 to 2.0 | 0.01 to 2.0 | 0.01 to 1.5 | 0.04 to 1.2 | 0.05 to 1.0 |
| Wax | 1.0 to 50 | 2.0 to 40 | 2.0 to 40 | 4.0 to 30 | 5.0 to 25 |
| C₁₆₋₁₈ fatty | 1.0 to 30 | 1.2 to 25 | 1.5 to 20 | 2.0 to 15 | 2.0 to 15 |
| alcohol | | | | | |
| Glycerol | 0.1 to 25 | 0.5 to 20 | 0.5 to 20 | 1.0 to 15 | 2.0 to 10 |
| Water | 5.0 to 90 | 10 to 85 | 40 to 85 | 65 to 82 | 65 to 82 |
| Optional | To 100 | To 100 | To 100 | To 100 | To 100 |
| additives | | | | | |

A second subject of the present invention is the use of a cosmetic composition of the invention for the temporary shaping of keratin-containing fibers, in particular human hair.

A third subject of the present invention is a method for the temporary shaping of keratin-containing fibers, in particular human hair, in which the keratinic fibers are acted upon by a cosmetic composition of the invention and are fixed temporarily in their shape.

The compositions, uses, and methods of the invention and some of their preferred embodiments are characterized by the following points:

1. Cosmetic compositions, including

a) 0.05 to 10% by weight of a hydrophobized metal oxide powder,

b) 0.01 to 2.0% by weight of at least one white pigment,

c) 1.0 to 50% by weight of wax,

d) 1.0 to 30% by weight of an emulsifier,

e) 0.1 to 25% by weight of an organic polyol, and

f) 5.0 to 90% by weight of water.

2. The compositions according to point 1, characterized in that they include the hydrophobized metal oxide powder, based on their total weight, in amounts of 0.1 to 10% by weight, preferably of 0.2 to 8.0% by weight, and in particular of 0.5 to 4.0% by weight.
3. The compositions according to one of the preceding points, characterized in that they include as the hydrophobized metal oxide powder a hydrophobized silicate obtained by silanization of pyrogenic silicon dioxide.
4. The compositions according to one of the preceding points, characterized in that they include white pigment b), based on their total weight, in amounts of 0.02 to 1.5% by weight, preferably of 0.04 to 1.2% by weight, and in particular of 0.05 to 1.0% by weight.
5. The compositions according to one of the preceding points, characterized in that they include as the white pigment a white pigment from the group comprising titanium oxide, zinc oxide, zinc sulfide, lithopone, and aluminum starch octenylsuccinate, preferably titanium oxide.
6. The compositions according to one of the preceding points, characterized in that they include wax c), based on their total weight, in amounts of 2.0 to 40% by weight, preferably of 4.0 to 30% by weight, and in particular of 5.0 to 25% by weight.
7. The compositions according to one of the preceding points, characterized in that they include as wax c) at least one wax from the group comprising beeswax, carnauba wax, candelilla wax, montan wax, and microcrystalline waxes, preferably beeswax.
8. The compositions according to one of the preceding points, characterized in that they include emulsifier d), based on their total weight, in amounts of 1.2 to 25% by weight, preferably of 1.5 to 20% by weight, and in particular of 2.0 to 15% by weight.
9. The composition according to one of the preceding points, characterized in that emulsifier d) is selected
i) from the group of fatty acid partial glycerides, preferably from the group of glycerol monostearate and glycerol distearate,
ii) from the group of surface-active, medium-chain fatty alcohols, preferably from the group of cetyl alcohol and stearyl alcohol
   10. The compositions according to one of the preceding points, characterized in that they include organic polyol e), based on their total weight, in amounts of 0.5 to 20% by weight, preferably of 1.0 to 15% by weight, and in particular of 2.0 to 10% by weight.
   11. The compositions according to one of the preceding points, characterized in that they include as organic polyol e) at least one polyol from the group comprising glycerol, polyethylene glycol, propylene glycol, butylene glycol, and hexanediol, preferably glycerol.
   12. The compositions according to one of the preceding points, characterized in that they include 10 to 85% by weight, preferably 40 to 85% by weight, and in particular 65 to 82% by weight of water.
   13. The compositions according to one of the preceding points, including

a) 0.5 to 4.0% by weight of a hydrophobized metal oxide powder,

b) 0.05 to 1.0% by weight of at least one white pigment,

c) 5.0 to 25% by weight of wax,

d) 2.0 to 15% by weight of an emulsifier,

e) 2.0 to 10% by weight of an organic polyol, and

f) 65 to 82% by weight of water.

14. The compositions according to one of the preceding points including
a) 0.5 to 4.0% by weight of a hydrophobized silicate obtained by silanization of pyrogenic silicon dioxide,
b) 0.05 to 1.0% by weight of titanium dioxide,
c) 5.0 to 25% by weight of beeswax,
d) 2.0 to 15% by weight of an emulsifier,
e) 2.0 to 10% by weight of glycerol, and
f) 65 to 82% by weight of water.
   15. The compositions according to one of the preceding points including

g) 0.1 to 10% by weight at least one homo- and/or copolymer.

16. The compositions according to one of the preceding points, that they include homo- or copolymer g), based on their total weight, in amounts of 0.2 to 10% by weight, preferably of 0.4 to 6.0% by weight, and in particular of 0.8 to 4.0% by weight.
17. The compositions according to one of the preceding points, characterized in that they include as the homo- and/or copolymer a homo- and/or copolymer from the group comprising polyvinylpyrrolidone and/or vinylpyrrolidone/vinyl acetate copolymer, preferably polyvinylpyrrolidone.
18. Use of a cosmetic composition according to one of the preceding points for the temporary shaping of keratin-containing fibers, in particular human hair.
19. A method for the temporary shaping of keratin-containing fibers, in particular human hair, in which the keratinic fibers are acted upon by a cosmetic composition according to one of points 1 to 17 and are temporarily fixed in their shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. Cosmetic compositions, comprising:
g) 0.05 to 10% by weight of a hydrophobized metal oxide powder,
h) 0.01 to 2.0% by weight of at least one white pigment,
i) 1.0 to 50% by weight of wax,
j) 1.0 to 30% by weight of an emulsifier,
k) 0.1 to 25% by weight of an organic polyol, and
l) 5.0 to 90% by weight of water.

2. The compositions according to claim 1, wherein the hydrophobized metal oxide powder comprises 0.1 to 8.0% by weight of the total composition.

3. The compositions according to claim 1, wherein the hydrophobized metal oxide powder comprises 0.2 to 6.0% by weight of the total composition.

4. The compositions according to claim 1, wherein the hydrophobized metal oxide powder comprises 0.5 to 4.0% by weight of the total composition.

5. The compositions according to claim 1, wherein the hydrophobized metal oxide powder is a hydrophobized silicate obtained by silanization of pyrogenic silicon dioxide.

6. The compositions according to claim 1, wherein the white pigment b) comprises 0.02 to 1.5% by weight of the total composition.

7. The compositions according to claim 1, wherein the white pigment b) comprises 0.04 to 1.2% by weight by weight of the total composition.

8. The compositions according to claim 1, wherein the white pigment b) comprises 0.05 to 1.0% by weight of the total composition.

9. The compositions according to claim 1, wherein the white pigment is selected from the group consisting of titanium oxide, zinc oxide, zinc sulfide, lithopone, and aluminum starch octenylsuccinate.

10. The compositions according to claim 1, wherein the white pigment includes titanium oxide.

11. The compositions according to claim 1, wherein the wax c) comprises 2.0 to 40% by weight of the total composition.

12. The compositions according to claim 1, wherein the wax c) comprises 4.0 to 30% by weight of the total composition.

13. The compositions according to claim 1, wherein the wax c) comprises 25.0 to 25% by weight of the total composition

14. The compositions according to claim 1, wherein the emulsifier d) comprises 1.2 to 25% by weight of the total composition.

15. The compositions according to claim 1, wherein the emulsifier d) comprises 1.5 to 20% by weight of the total composition.

16. The compositions according to claim 1, wherein the emulsifier d) comprises 2.0 to 15% by weight of the total composition.

17. The compositions according to claim 1, wherein the organic polyol e) comprises 0.5 to 20% by weight of the total composition.

18. The compositions according to claim 1, wherein the organic polyol e) comprises 1.0 to 15% by weight of the total composition.

19. The compositions according to claim 1, wherein the organic polyol e) comprises 2.0 to 10% by weight of the total composition.

20. A method for the temporary shaping of keratin-containing fibers, in which the keratinic fibers are acted upon by a cosmetic composition according to claim 1 and are temporarily fixed in their shape.
